(19) United States
(12) Patent Application Publication
(10) Pub. No.: US 2016/0263374 A1
(43) Pub. Date: Sep. 15, 2016

(54) SIMULATION OF ATMOSPHERIC ELECTRIC FIELD

(71) Applicant: Zhen LI, Toronto (CA)

(72) Inventor: ZHEN LI, TORONTO (CA)

(21) Appl. No.: 15/030,374

(22) PCT Filed: Apr. 28, 2014
(86) PCT No.: PCT/IB2014/061056

§ 371 Date: Apr. 18, 2016

(30) Foreign Application Priority Data
Oct. 21, 2013 (CA) 2830539
Nov. 13, 2013 (CA) 2832764

(51) Int. Cl. A61N 1/20; A61B 5/00; A61H 39/00; A61M 21/02; A61H 39/08

(52) U.S. Cl. CPC A61N 1/20; A61M 21/02; A61H 39/08; A61H 39/002; A61B 5/4854; A61M 2021/0055

(57) ABSTRACT

This invention relates to simulate an electric field which is relatively stable and favorable for living creature. It belongs to the field of bioengineering/electronic device.

### BACKGROUND

Existing inventions has some disadvantages: 1. It applies an AC (alternating current) electric field. 2. Or apply a very high voltage DC (direct current) electric field, for example:CN 1309889, which is not respect the strength, of fair weather atmospheric electric field 3. Or apply DC electric field with an short cycle impulse. 4. Or apply DC electric field without certain strength, only the voltage is set but the strength of electric field is not set, strength might vary significantly. 5 . Or apply DC electric field without detecting/controlling the strength of the electric field, the electric field might overlap with other electric field nearby thus the strength might vary significantly. 6. Or apply negative DC electric field.

Disadvantages of these inventions are against natural of creature, are not favorable for living creature, and might even be harmful to living creature.

Object of Invention

On the other hand, this invention generating and detecting and controlling a low voltage positive DC (direct circuit) electric field without impulse; its strengh is simular as atmospheric fair weather electric field, saying less than 300 V/m.

Description

Modern technologies bring us a better life, but in the same time bring contaminations to our living environment. These contaminations do not only include air, water or soil pollution, but also include electric field pollution, and electromagnetic radiation. Also there are some factors in nature environment that disturber creature in the earth. To shield these contaminations or noise and to create a clean and quiet atmospheric electric field is the key to recover from diseases, bring wellness to our life.

Under fair weather condition, living creature has a better metabolism. On the other hand, bad weather like storms, rain, overcast, especially thunder-storms, affects the nervous system of creature unpleasantly, and brings on or aggravates neuralgic, rheumatic and other pains, as well as incites mental distress and discouragement, because there are irregular disturbances in the electrical condition of the atmosphere. Weather condition is closely related with health condition of living creature. Under a fair weather condition, atmospheric electricity potential (voltage) increases about 150 V/m when climbing against the gradient of the electric field; under a bad weather condition, the electricity potential (voltage) decrease, or turns to negative, or rapidly changes.

Modern technologies using a lot metal in our environment, like steel in building structure, steel roof, copper wires, lightning rod. Metal is good conductor, it shields atmospheric electric field, thus indoor creature always live without positive electric field, like living in bad weather condition.

In the environment that creature adopt for million and million years, relatively stable of atmospheric electric field can be considered as a benefit. But smoothly and regularly change can be considered as one kind of tide energy, creatures benefit from these smoothly and regularly change. This atmospheric electric field is modulated weakly by solar diurnal cycle, annual cycle, by monthly cycle, seasonally cycle, 10 yearly cycle, 12 yearly cycle, 60 yearly cycle, 180 yearly cycle, and 15 minutes cycle.

### EXAMPLE

Raymond, had been felt distention in right side of middle abdomen for more than 10 years, was diagnoses as liver cancer last year. He was waiting for live transplant. After used this device for 2 weeks, distention feeling had been reduced to 30% of before. After use for 3 months, the index of cancer has been lowered significantly.

Jessie, had constipation for more than 7 years, defecates every 3˜4 days, after using this device for 3 days, Her intestines moved much better, defecated very morning.

Elaine, was very skinny with poor appetites. 1 month after using the device, her appetites had been improved a lot and she gain 5 Kg.

Frank, have hypertension for 5 years, need to take pill to low the pressure, after 2 months, his blood pressure get normal without pills.

### BRIEFLY DESCRIBE FIGURES

FIG. 1: This figure states the block diagram of this system, its input is a DC supply, a micro computer unit monitors and control the out put.

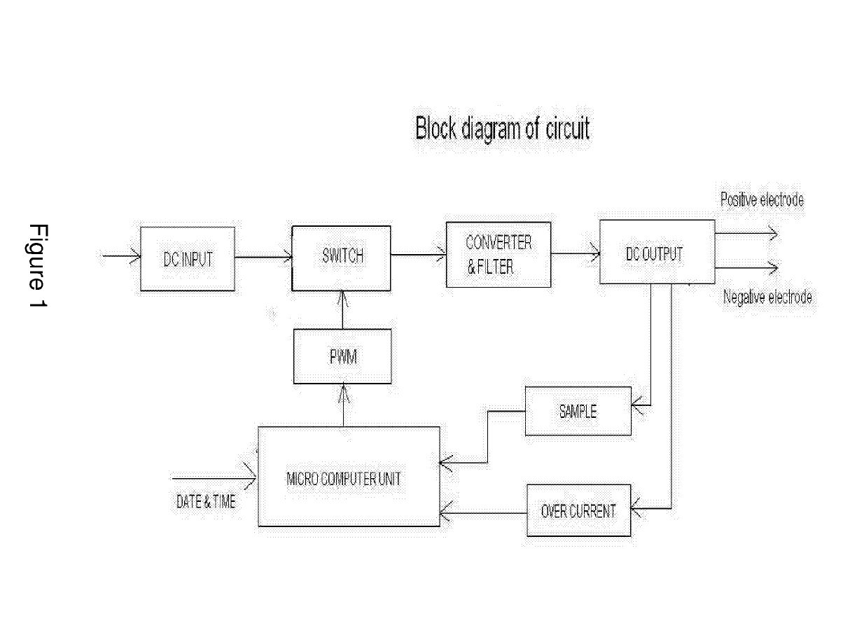

FIG. 2: A sample for installation, which is installed in a room.

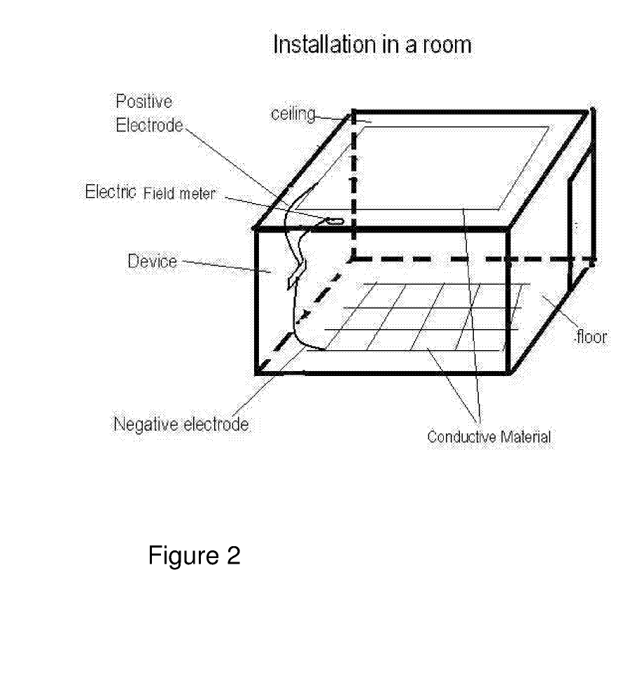

FIG. 3: Part of this figure is a sample of installation, which is installed in a bed. The rest is a mistake, it repeats of FIG. 1 and FIG. 2.

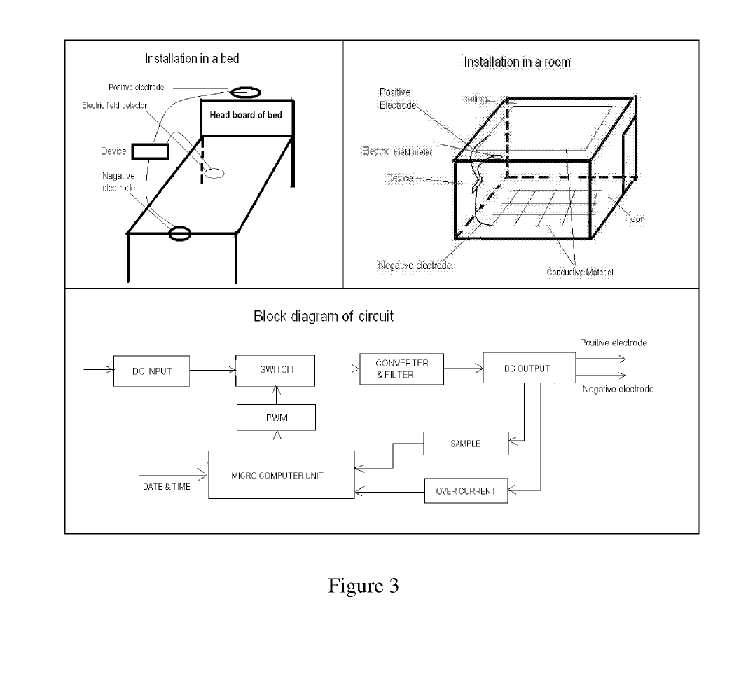

The embodiments of the invention in which an exclusive property or privilege is claimed are defined as follows:

1. A process for generating a positive electric field in a room, comprising generating certain strength of DC electric field for human, in a room or surrounding material, like walls, beds, roof, floor, chairs, car, footwear, headwear, gloves, or clothing.

2. A process for generating a positive electric field in a room, comprising generating certain strength of DC electric field for other purpose except for human, in a room or surrounding material, like walls, beds, roof, floor, chairs, car, footwear, headwear, gloves, or clothing.

3. A process for generating electric field in claims 1,2, comprising generating and detecting and controlling certain strength of DC electric field.

4. A process for generating electric field in a room as claims 1,2,3, which strength is less than 300 V/m.

5. A process for generating electric field in a room as claims 1,2,3,4, which is modulated by solar diurnal cycle, annual cycle, by monthly cycle, seasonally cycle, 10 yearly cycle, 12 yearly cycle, 60 yearly cycle, 180 yearly cycle, and 15+minutes cycle.

6. A process for generating electric field in a room as claims 1,2,3,4,5, two electrodes can touched or not touched by users.

7. A process for generating electric field in a room as claims 1,2,3,4,5, two electrodes can be any shape, any conductive material, any semi-conductive material.

8. A device for generating electric field in a room as claims 1,2,3,4,5,6,7.

9. A method comprising receiving acupuncture/massage treatment when clients are in electric field generated by these devices as claims 1,2,3,4,5,6,7,8.

10. A method comprising the acupuncturist/other health practitioner being in electric field generated by these devices as claims 1,2,3,4,5,6,7,8 when he/she do acupuncture or other treatment for other people.

11. A method comprising connecting the positive electrode of devices as claims 1,2,3,4,5,6,7,8 to relatively positive portion of body, and negative electrode to relatively negative portion of body.

12. A method comprising person being in electric field generated by these devices as claims 1,2,3,4,5,6,7,8 when he/she is practicing Tai Chi or Qigong.
